(19) United States
(12) Patent Application Publication
(10) Pub. No.: US 2022/0287891 A1
(43) Pub. Date: Sep. 15, 2022

(54) ABSORBENT ARTICLE HAVING APERTURED SIDE PANELS

(71) Applicant: KIMBERLY-CLARK WORLDWIDE, INC, Neenah, WI (US)

(72) Inventors: SeungKeun PARK, Guri-si (KR); EoYeon HWANG, YongIn-si (KR)

(21) Appl. No.: 17/439,847

(22) PCT Filed: Mar. 30, 2019
(86) PCT No.: PCT/US2019/025068

§ 371 Date: Sep. 16, 2021

(51) Int. Cl. A61F 13/49; A61F 13/15

(52) U.S. Cl. CPC A61F 13/49015; A61F 13/15203; A61F 2013/15552

(57) ABSTRACT

A new disposable absorbent article having a plurality of slits positioned in a middle region below the top and above the bottom edges of side panels of the absorbent article has been discovered. By placing the plurality of slits below the top and above the bottom edges of the side panels improve absorbent article breathability and lessens the amount of force the absorbent article applies onto the wearer's leg when donned.

### BACKGROUND OF THE DISCLOSURE

Infants and other incontinent individuals wear disposable absorbent articles such as diapers to receive and contain urine and other body exudates. In order to contain body exudates as well as to fit a wide variety of body shapes and sizes, these pants must fit snugly about the waist and legs of the wearer without drooping, sagging or sliding down from its position on the torso as well as fitting larger wearers without causing irritation to the skin due to the product being too tight. Thus, the pant must have elastic extensibility in the waist and legs with the elastic features providing a high degree of stretch.

Many absorbent articles such as diapers, training pants, incontinence products, other personal care or health care garments or the like use conventional elastic elements secured in an elastically contractible condition in the waist and leg openings. Typically, in order to insure full elastic fit about the leg and the waist such as is provided with durable undergarments, the leg openings and waist opening are encircled with elasticized bands of rubber or other materials positioned along the curve of the opening. These pants are typically characterized as “balloon style” pants because of the contraction caused by the elasticized bands in specific zones of the product while the remaining material tends to blouse. Examples of such training pants are disclosed in U.S. Pat. No. 5,171,239 to lgaue, et al. on Dec. 15, 1992 and U.S. Pat. No. 4,610,681 to Strohbeen, et al. on Sep. 9, 1986. Although these pants will allow fit of various waist and leg sizes due to the fact that the contractive elastic openings will expand to accommodate various size wearers, these products still fit a limited range of fit sizes because the elastic elements are in contraction and do not have a high degree of stretch. The narrow elastic bands used in the waist opening and the leg openings also tend to concentrate the “fitment” forces in a narrow zone of the wearer's body leading to increased incidence of skin marking of the wearer. Further, the sides and remainder of the products are typically not elastically extensible thereby reducing the fit.

In order to solve this deficiency in balloon-style pants, some manufacturers have positioned elastic strands across the entire front and back regions of the product. For example, WO 93/17648 published on Sep. 16, 1993, discloses a pant-type diaper in which the front and/or the rear pans are elastically contractible and the ends of the absorbent core are disposed in these regions. These additional elastic strands act to contract the entire front and back waist parts of the pant including the end areas of the absorbent core. This allows greater expansion of the product in the waist region but causes the absorbent core to be gathered and bunch at the waist opening. This gathering and bunching results in a higher risk of leakage at the waist since channels are created along the absorbent core that allows body exudates to wick or flow out of the waist. This gathering of the absorbent core also affects the appearance of the product in that the product does not appear as aesthetically pleasing as the balloon style pants.

Another type training pant is shown in U.S. Pat. No. 4,940,464 issued to Van Gompel, et al. on Jul. 10, 1990, wherein a pant-like garment is formed by attaching discrete stretchable members to the side edges of the main body of the garment. This stretchable member allows the article to fit variations in size as the stretchable member expands to meet the size of the wearer. However, the seaming of the main body with the stretchable members are a major problem in the product and in the manufacturing process. The seams must be made extremely strong and capable of handling great forces during application and use. A pant undergoes severe forces and stresses during use which may cause the side panels to break away or tear from the main body portion. Further, controlling these separate stretchable members severely complicates the manufacturing process and causes these pants to be more costly to the consumer. The seams to the main body portion also tend to concentrate the fitment forces at the top and bottom of the product resulting in increased seam tearing and poorer fit of the garment.

U.S. Pat. No. 5,246,433 issued to Hasse, Bridges and Miller on Sep. 21, 1993, discloses a pant having stretch laminate side panels as well as elastic waistbands and leg bands. The stretch laminates in the side panels provide stretch in the sides of the product to better fit the pant on the wearer. The stretch laminates are unitary with the rest of the pant to overcome the drawbacks associated with the pants having separate and discrete elastic panel members attached thereto while improving the fit over the conventional balloon-style pants. However, these pants suffer from the drawback that the side panels are not sufficiently stretchable to allow a wearer to easily pull the pant on by themselves. The stretch laminates disclosed therein provide a limited amount of stretch. Further, the materials used to form the stretch laminates can have problems with tearing or shredding during use resulting in the products being scrapped by the manufacturer.

Another drawback with all of these products is the breathability of the product. The panels of the pant are typically formed by films or forms which can preclude the breathability of such products. This lack of breathability may result in a hot stuffy product for the wearer.

It is, therefore, desirable to provide a disposable absorbent article with a high degree of breathability at the side panels to allow vapor transmission so that the absorbent article is not hot and stuffy. Additionally it is desirable to provide a disposable absorbent article such that it will have improved stretchability and fit at the side panels so as to reduce waist and leg load tensions for the wearer.

### SUMMARY OF THE DISCLOSURE

In response to the above-referenced unfulfilled need in the art, a new disposable absorbent article having a plurality of slits positioned in a middle region below the top and above bottom edges of side panels of the absorbent article has been discovered. By placing the plurality of slits below the top and above the bottom edges of the side panels improves absorbent article breathability and lessens the amount of force the absorbent article applies onto the wearer's leg when donned.

In one embodiment, the present invention pertains to an absorbent article including an absorbent chassis defining: a longitudinal axis, a transverse axis, front and back waist edges parallel to the transverse axis, opposite side edges extending between the front and back waist edges, a front waist region contiguous with the front waist edge, a back waist region contiguous with the back waist edge, and a crotch region which extends between and interconnects the front and back waist regions, the front waist region defining a pair of transversely opposed front side panels having an upper edge contiguous with the front waist edge and a bottom edge and a front center panel positioned between and interconnecting the front side panels, the back waist region defining a pair of transversely opposed back side panels having an upper edge contiguous with the back waist edge and a bottom edge and a back center panel positioned between and interconnecting the back side panels, wherein the front and back side panels have a length extending from the upper edge to the lower edge, an upper portion adjacent to the upper edge and having an upper portion length, a lower portion adjacent to the bottom edge and having a lower portion length, and a middle portion disposed between the upper and lower portions and having a middle portion length a plurality of slits disposed within the middle portion and wherein the upper and lower portions are substantially free from slits.

In a further embodiment of the present invention, an absorbent article comprises: a chassis having a longitudinal axis, a transverse axis, a front waist portion having a front waist edge and a back waist portion having a back waist edge; a pair of elastomeric front side panels that are extensible in the transverse direction, the front side panels extending from the front waist portion and having an upper edge portion that is contiguous with the front waist edge, and a lower edge portion, an outer most surface and opposed inner most surface, each front side panel having a length extending from the upper edge to the lower edge, an upper portion adjacent to the upper edge and having an upper portion length, a lower portion adjacent to the bottom edge and having a lower portion length, and a middle portion disposed between the upper and lower portions and having a middle portion length, wherein the middle portion comprises a plurality of longitudinally orientated slits extending from the outer most surface to the inner most surface and having a first aspect ratio and the upper and lower portions are substantially free from slits; and a pair of elastomeric back side panels that are extensible in the transverse direction, the front side panels extending from the back waist portion and having an upper edge portion that is contiguous with the back waist edge, and a lower edge portion, an outer most surface and opposed inner most surface, each back side panel having a length extending from the upper edge to the lower edge, an upper portion adjacent to the upper edge and having an upper portion length, a lower portion adjacent to the bottom edge and having a lower portion length, and a middle portion disposed between the upper and lower portions and having a middle portion length, wherein the middle portion comprises a plurality of longitudinally orientated slits extending from the outer most surface to the inner most surface and having a first aspect ratio and the upper and lower portions are substantially free from slits, wherein upon donning of the article by a wearer the first aspect ratio is decreased.

In yet a further embodiment of the present invention discloses a side panel for joining the front waist region and rear waist region of an absorbent article chassis having a longitudinal axis and a transverse axis, the side panel comprising: an elastic laminate extensible in the transverse direction and comprising two layers of nonwoven on either side of an elastic film, the laminate having first and second opposed outer surfaces; an upper edge; a lower edge; a length extending from the upper edge to the lower edge; an upper portion adjacent to the upper edge and having an upper portion length and being substantially free from apertures, a lower portion adjacent to the bottom edge and having a lower portion length and being substantially free from apertures, and a middle portion disposed between the upper and lower portions and having a middle portion length and having a plurality of longitudinally orientated slits extending from the first to the second outer surfaces and having a first aspect ratio.

### BRIEF DESCRIPTION OF DRAWINGS

The disclosure may be more completely understood in consideration of the following detailed description of various embodiments in connection with the accompanying drawing, in which:

FIG. 1 illustrates a side view of one type of disposable absorbent article incorporating the principles of the present disclosure, where the front and back side panels have slits.

FIG. 2 illustrates a plan view of the disposable absorbent article shown in FIG. 1 in an open, stretched and laid flat condition, and showing the surface of the article that faces away from the wearer.

FIG. 3 illustrates a side view similar to FIG. 1, but illustrating the front and back side panels in an extended state when donned by the wearer.

FIG. 4A illustrates a linear slit in a non-extended state.

FIG. 4B illustrates a hole in an extended state.

FIG. 5A illustrates a curvilinear slit in a non-extended state.

FIG. 5B illustrates an alternative type of hole in an extended state.

FIG. 6A illustrates an alternative type of slit in a non-extended state.

FIG. 6B illustrates an alternative type of hole in an extended state.

FIG. 7A illustrates an alternative type of slit in a non-extended state.

FIG. 7B illustrates an alternative type of hole in an extended state.

FIGS. 8A-8D illustrate different patterns and holes in an extended state on a side panel.

FIG. 9 illustrates a pattern of holes in an extended state on a side panel.

FIG. 10 illustrates a pattern of holes in an extended state on a side panel.

FIG. 11 illustrates extension and recovery force curves for two cycle hysteresis of side panels.

While the disclosure is amenable to various modifications and alternative forms, specifics thereof have been shown by way of example in the drawing and will be described in detail. It should be understood, however, that the intention is not to limit aspects of the disclosure to the particular embodiments described. On the contrary, the intention is to cover all modifications, equivalents, and alternatives falling within the scope of the disclosure.

### DEFINITIONS

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements.

The term “absorbent article” refers to devices that absorb and contain body exudates, and, more specifically, refers to devices that are placed against or in proximity to the body of the wearer to absorb and contain the various exudates discharged from the body. Absorbent articles may include diapers, pant diapers, open diapers, diaper covers having fastening means for fastening the diaper, training pants, adult incontinence undergarments, feminine hygiene products, breast pads, care mats, bibs, wound dressing products, and the like. As used herein, the term “body exudates” includes, but is not limited to, urine, blood, vaginal discharges, breast milk, sweat and fecal matter.

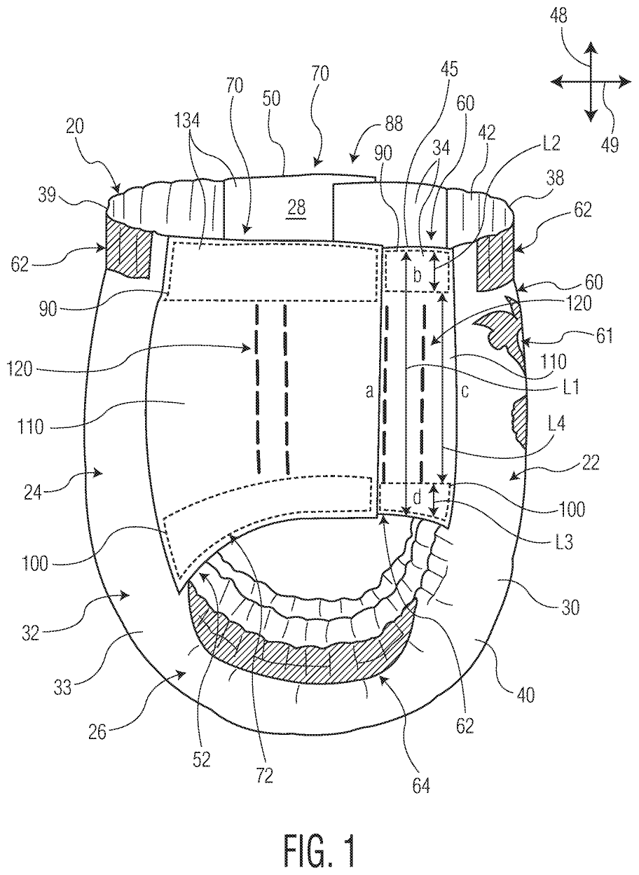

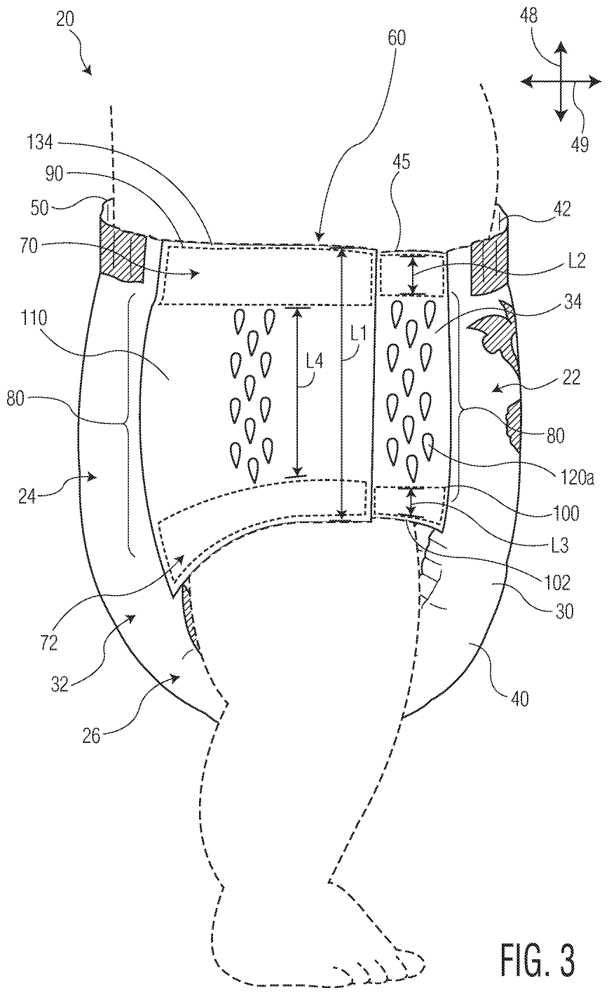

The term “longitudinal” and “transverse” have their customary meaning, as indicated by the longitudinal and transverse axes depicted in FIGS. 1 and 3. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

The term “tension” includes a uniaxial force tending to cause the extension of a body or the balancing force within that body resisting the extension.

The term a “layer” or “web” as used herein is a generally two-dimensional structure that may comprise one or more plies and may be in the form of a laminate made from plies of the same or different materials.

The term “aspect ratio” when referring to the dimensions of an aperture, such as a slit or a hole, is the ratio of the lengths of the major and minor axis. For example, with reference to FIG. 1 the hole (120a) has a major axis (48) having a first length and a minor axis (49) having a second length where the ratio of the first length to the second length is the aspect ratio of the hole (120a).

The term “aperture” refers to gap, hole, orifice or other opening extending form a first surface to a second surface.

The term “slit” refers to an aperture having an aspect ratio greater than about 3, such as greater than 5, such as greater than 10. In certain embodiments, slits may be selectively disposed in the middle portion of a side panel and form a continuous passage from the a first outer surface of the panel to a second outer surface and may have a relatively high aspect ratio, such as greater than about 3, such as greater than 5, such as greater than 10 and a small open area when the side panel is in an untensioned state.

The term “hole” refers to an aperture having an aspect ratio less than 3, such as less than 2. In certain embodiments, holes may be selectively disposed in the middle portion a side panel and form a continuous passage from the a first outer surface of the panel to a second outer surface and may have an aspect ratio less than 3, such as less than 2, such as from about 0.5 to about 1.5 and have an open area from about 2 mm to about 5 mm and from about 3 mm to about 5 mm when in a tensioned state. The dimensions or a hole are generally measured when the article or side panel is tensioned, such as an extension force of about 700 gf.

As used herein, the term “extensible” refers to any material which, upon application of a biasing force, is elongatable by at least about 10 percent, at least about 20, or at least about percent 50 percent, without experiencing catastrophic failure. Recovery of the strain is not required for a material to be considered extensible.

### DETAILED DESCRIPTION

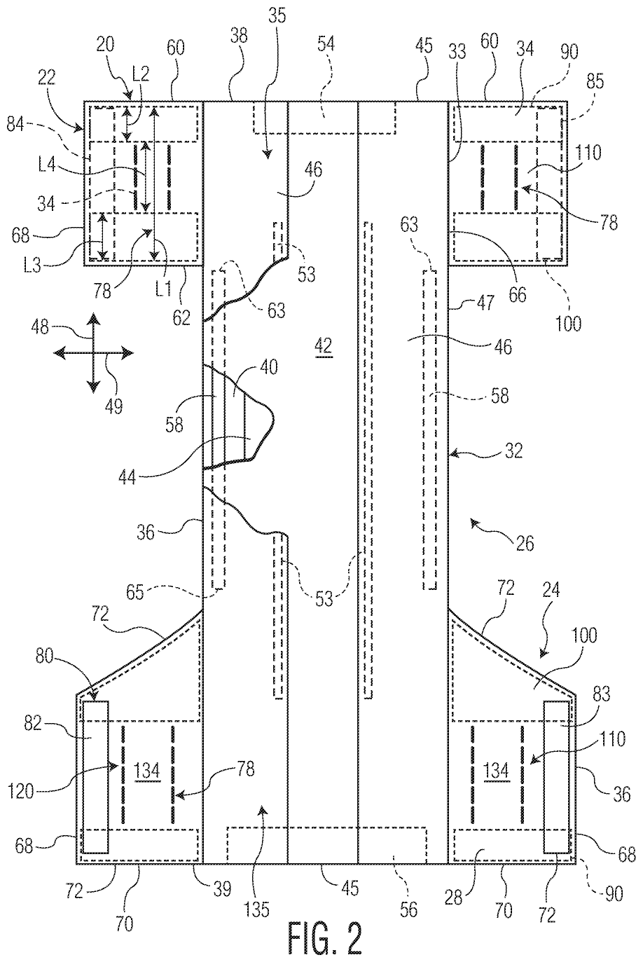

With reference to FIG. 1, a disposable absorbent article, such as a diaper pant 20, is illustrated in a non-extended state wherein the front and back side panels 34, 134 have slits 120. The diaper pant 20 comprises an absorbent chassis 32. The absorbent chassis 32 defines a front waist region 22, a back waist region 24, a crotch region 26 interconnecting the front and back waist regions, an inner surface 28 which is configured to contact the wearer, and an outer surface 30 opposite the inner surface which is configured to contact the wearer's clothing. With additional reference to FIG. 2, the absorbent chassis 32 also defines a pair of transversely opposed side edges 36 and a pair of longitudinally opposed waist edges, which are designated front waist edge 38 and back waist edge 39. The front waist region 22 is contiguous with the front waist edge 38, and the back waist region 24 is contiguous with the back waist edge 39.

The illustrated absorbent chassis 32 comprises a rectangular composite structure 33, a pair of transversely opposed front side panels 34, and a pair of transversely opposed back side panels 134. The composite structure 33 and side panels 34 and 134 may be integrally formed or comprise two or more separate elements, as shown in FIGS. 1 and 3. The illustrated composite structure 33 comprises an outer cover 40, a bodyside liner 42 (FIGS. 1-3) which is connected to the outer cover in a superposed relation, an absorbent assembly 44 (FIG. 3) which is located between the outer cover and the bodyside liner, and a pair of containment flaps 46 (FIG. 2). The rectangular composite structure 33 has opposite linear end edges 45 that form portions of the front and back waist edges 38 and 39, and opposite linear side edges 47 that form portions of the side edges 36 of the absorbent chassis 32 (FIG. 2). For reference, arrows 48 and 49 depicting the orientation of the longitudinal axis and the transverse axis, respectively, of the diaper pant 20 are illustrated in FIG. 1.

The front and back waist regions 22 and 24 of the diaper pant 20 are joined together to define a three-dimensional pant configuration having a waist opening 50 and a pair of leg openings 52 (FIG. 1). The front waist region 22 comprises the portion of the diaper pant 20 which, when worn, is positioned on the front of the wearer while the back waist region 24 comprises the portion of the diaper pant which, when worn, is positioned on the back of the wearer. The crotch region 26 of the training pant 20 comprises the portion of the diaper pant 20 which, when worn, is positioned between the legs of the wearer and covers the lower torso of the wearer. The front and back side panels 34 and 134 comprise the portions of the diaper pant 20 which, when worn, are positioned on the hips of the wearer.

The front waist region 22 of the absorbent chassis 32 includes the transversely opposed front side panels 34 and a front center panel 35 (FIGS. 2 and 3) positioned between and interconnecting the side panels. The back waist region 24 of the absorbent chassis 32 includes the transversely opposed back side panels 134 (FIGS. 1 and 3) positioned between and interconnecting the side panels.

The absorbent chassis 32 is configured to contain and/or absorb any body exudates discharged from the wearer. For example, the absorbent chassis 32 may, although not necessarily, comprise the pair of containment flaps 46 which are configured to provide a barrier to the transverse flow of body exudates. A flap elastic member 53 (FIG. 2) is operatively joined with each containment flap 46 in any suitable manner as is well known in the art. The elasticized containment flaps 46 define an unattached edge which assumes an upright, generally perpendicular configuration in at least the crotch region 26 of the diaper pant 20 to form a seal against the wearer's body. The containment flaps 46 may be located along the transversely opposed side edges of the absorbent chassis 32, and can extend longitudinally along the entire length of the absorbent chassis or may only extend partially along the length of the absorbent chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pant 20 desirably includes a front waist elastic member 54, a rear waist elastic member 56, and leg elastic members 58, as are known to those skilled in the art (FIG. 3). The waist elastic members 54 and 56 can be operatively joined to the outer cover 40 and/or bodyside liner 42 along the opposite waist edges 38 and 39, and can extend over part or all of the waist edges. The leg elastic members 58 are desirably operatively joined to the outer cover 40 and/or bodyside liner 42 along the opposite side edges 36 and positioned in the crotch region 26 of the training pant 20. The leg elastic members 58 are desirably longitudinally aligned along each side edge 47 of the composite structure 33. Each leg elastic member 58 has a front terminal point 63 and a back terminal point 65, which points represent the longitudinal ends of the elastic gathering caused by the leg elastic members. The front terminal points 63 are desirably located adjacent the longitudinally innermost parts of the front side panels 34, and the back terminal points 65 are desirably located adjacent the longitudinally innermost parts of the back side panels 134.

The flap elastic members 53, the waist elastic members 54 and 56, and the leg elastic members 58 (FIG. 2) may be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat; such that elastic constrictive forces are imparted to the substrate. In one particular embodiment, for example, the leg elastic members 58 comprise a plurality of dry-spun coalesced multifilament spandex elastomeric threads sold under the trade name LYCRA(R) and available from E. I. Du Pont de Nemours and Company, Wilmington, Del., U.S.A.

The outer cover 40 desirably comprises a material that is substantially liquid impermeable, and can be elastic, stretchable or nonstretchable. The outer cover 40 may be a single layer of liquid impermeable material, but desirably comprises a multi-layered laminate structure in which at least one of the layers is liquid impermeable. For instance, the outer cover 40 may include a liquid permeable outer layer and a liquid impermeable inner layer that are suitably joined together by a laminate adhesive (not shown). Suitable laminate adhesives, which may be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture. One example of such a material is a 20 gsm (grams per square meter) spunbond polypropylene nonwoven web. The outer layer may also be made of those materials of which liquid permeable bodyside liner 42 is made. While it is not a necessity for outer layer to be liquid permeable, it is desired that it provides a relatively cloth-like texture to the wearer.

The inner layer of the outer cover 40 may be both liquid and vapor impermeable, or can be liquid impermeable and vapor permeable. The inner layer is desirably manufactured from a thin plastic film, although other flexible liquid impermeable materials may also be used. The inner layer, or the liquid impermeable outer cover 40 when a single layer, prevents waste material from wetting articles, such as bedsheets and clothing, as well as the wearer and caregiver. A suitable liquid impermeable film for use as a liquid impermeable inner layer, or a single layer liquid impermeable outer cover 40, is a 1.0 mil polyethylene film commercially available from Edison Plastics Company of South Plainfield, N.J., U.S.A. If the outer cover 40 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer cover 40. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

As shown in FIGS. 1-3, the diaper pant 20 and in particular the outer cover 40 desirably comprises one or more appearance-related components. Examples of appearance-related components include, but are not limited to, graphics; highlighting or emphasizing leg and waist openings in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product.

The liquid permeable bodyside liner 42 is illustrated as overlying the outer cover 40 and may but need not have the same dimensions as the outer cover 40. The bodyside liner 42 is desirably compliant, soft feeling, and non-irritating to the child's skin.

The bodyside liner 42 may be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics may be used for the bodyside liner 42. For example, the bodyside liner may be composed of a meltblown or spunbonded web of polyolefin fibers. The bodyside liner may also be a bonded-carded web composed of natural and/or synthetic fibers. The bodyside liner may be composed of a substantially hydrophobic material, and the hydrophobic material can, optionally, be treated with a surfactant or otherwise processed to impart a desired level of wettability and hydrophilicity. For example, the material can be surface treated with about 0.28 weight percent of a surfactant commercially available from the Rohm and Haas Co. under the trade designation Triton X-102. The surfactant can be applied by any conventional means, such as spraying, printing, brush coating or the like. The surfactant can be applied to the entire bodyside liner 42 or can be selectively applied to particular sections of the bodyside liner, such as the medial section along the longitudinal centerline.

A suitable liquid permeable bodyside liner 42 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like. While the outer cover 40 and bodyside liner 42 can comprise elastomeric materials, it can be desirable in some embodiments for the composite structure to be generally inelastic, where the outer cover, the bodyside liner and the absorbent assembly comprise materials that are generally not elastomeric.

The absorbent assembly 44 (FIG. 2) is positioned between the outer cover 40 and the bodyside liner 42, which components may be joined together by any suitable means such as adhesives as is well known in the art. The absorbent assembly 44 may be any structure which is generally compressible, conformable, non-irritating to the baby or child's skin, and capable of absorbing and retaining liquids and certain body wastes. The absorbent assembly 44 may be manufactured in a wide variety of sizes and shapes, and from a wide variety of liquid absorbent materials commonly used in the art. For example, the absorbent assembly 44 may suitably comprise a matrix of hydrophilic fibers, such as a web of cellulosic fluff, mixed with particles of a high-absorbency material commonly known as superabsorbent material. In a particular embodiment, the absorbent assembly 44 comprises a matrix of cellulosic fluff, such as wood pulp fluff, and superabsorbent hydrogel-forming particles. The wood pulp fluff can be exchanged with synthetic, polymeric, meltblown fibers or with a combination of meltblown fibers and natural fibers. The superabsorbent particles can be substantially homogeneously mixed with the hydrophilic fibers or can be nonuniformly mixed. The fluff and superabsorbent particles can also be selectively placed into desired zones of the absorbent assembly 44 to better contain and absorb body exudates. The concentration of the superabsorbent particles can also vary through the thickness of the absorbent assembly 44. Alternatively, the absorbent assembly 44 may comprise a laminate of fibrous webs and superabsorbent material or other suitable means of maintaining a superabsorbent material in a localized area.

Suitable superabsorbent materials may be selected from natural, synthetic, and modified natural polymers and materials. The superabsorbent materials can be inorganic materials, such as silica gels, or organic compounds, such as crosslinked polymers. Suitable superabsorbent materials are available from various commercial vendors, such as Dow Chemical Company located in Midland, Mich., U.S.A., and Stockhausen GmbH and Co. KG, D-47805 Krefeld, Federal Republic of Germany. Typically, a superabsorbent material is capable of absorbing at least about 15 times its weight in water, and desirably is capable of absorbing more than about 25 times its weight in water.

In one embodiment, the absorbent assembly 44 is generally rectangular in shape, and comprises a blend of wood pulp fluff and superabsorbent material. One preferred type of fluff is identified with the trade designation CR1654, available from Kimberly-Clark Corporation, Neenah, Wis., U.S.A., and is a bleached, highly absorbent sulfate wood pulp containing primarily soft wood fibers. As a general rule, the superabsorbent material is present in the absorbent assembly 44 in an amount of from about 5 to about 90 weight percent based on total weight of the absorbent assembly. The absorbent assembly 44 suitably has a density within the range of about 0.10 to about 0.35 grams per cubic centimeter. The absorbent assembly 44 may or may not be wrapped or encompassed by a suitable tissue wrap that maintains the integrity and/or shape of absorbent assembly.

The absorbent chassis 32 may also incorporate other materials that are designed primarily to receive, temporarily store, and/or transport liquid along the mutually facing surface with absorbent assembly 44, thereby maximizing the absorbent capacity of absorbent assembly. One suitable material is referred to as a surge layer (not shown) and comprises a material having a basis weight of about 50 grams per square meter, and comprising a through-air-bonded-carded web of a homogenous blend of 60 percent 3 denier bicomponent fiber comprising a polyester core/polyethylene sheath, commercially available from BASF Corporation, and 40 percent 6 denier polyester fiber, commercially available from Hoechst Celanese Corporation, in Portsmouth, Va., U.S.A.

As noted previously, the illustrated diaper pant 20 has front and back side panels 34 and 134 disposed on each side of the absorbent chassis 32. These transversely opposed front side panels 34 and transversely opposed back side panels 134 can be permanently bonded to the composite structure 33 of the absorbent chassis 32 in the respective front and back waist regions 22 and 24, and are releasably attached to one another by the fastening system 80. More particularly, as shown best in FIG. 2, the front side panels 34 may be permanently bonded to and extend transversely beyond the linear side edges 47 of the composite structure 33 in the front waist region 22 along attachment lines 66, and the back side panels 134 may be permanently bonded to and extend transversely beyond the linear side edges of the composite structure in the back waist region 24 along attachment lines 66. The side panels 34 and 134 may be attached using attachment means known to those skilled in the art such as adhesive, thermal or ultrasonic bonding. The side panels 34 and 134 may also be formed as a portion of a component of the composite structure 33, such as the outer cover or the bodyside liner.

The illustrated side panels 34 and 134 each define a distal edge 68 that is spaced from the attachment line 66, a waist end edge 72 disposed toward the longitudinal center of the training pant 20, and a waist end edge 72 disposed toward a longitudinal end of the training pant. The waist end edge 72 extend from the side edges 47 of the composite structure 33 to the distal edges 68. The waist end edges 72 of the side panels 34 and 134 form part of the side edges 36 of the absorbent chassis 32. In the back waist region 24, the waist end edges 72 are desirably although not necessarily angled relative to the transverse axis 49 to provide greater coverage toward the back of the pant as compared to the front of the pant. The waist end edges 72 are desirably parallel to the transverse axis 49. The waist end edges 72 of the front side panels 34 form part of the front waist edge 38 of the absorbent chassis 32, and the waist end edges 72 of the back side panels 134 form part of the back waist edge 39 of the absorbent chassis.

The side panels 34 and 134 desirably have a length dimension (L1) measured parallel to the longitudinal axis 48 from the front upper edge 60 to the front bottom edge 62. The length dimension L1 may vary, however, in certain preferred embodiments L1 is at least about 20 percent, such as at least about 25 percent, such as about 30 percent or greater, of the overall length dimension of the absorbent article, also measured parallel to the longitudinal axis 48.

As shown in FIGS. 1 and 2, in an exemplary embodiment, the longitudinal distance L1 of the first and second back side panels 134 are greater than the longitudinal distance L1 of the first and second front side panels 34. In certain embodiments, the longitudinal distance L1 of the first and second back side panels may be from about 1 to about 2.5 times greater than the longitudinal distance L1 of the first and second front side panels. Further, while illustrated as substantially equivalent lengths, the longitudinal distance L1 for a given pair of panels may be different lengths.

The side panels are generally rectilinear in shape, however, they may assume any number of different shapes and the shape of the panels may differ from one another. For example, the shape of the first and second back side panel may differ from the shape of the first and second front side panel, such as in the embodiments illustrated in FIGS. 1 and 2. In those instance where a side panel is not rectangular, such as the first and second back side panels 134 of FIGS. 1 and 2, the longitudinal distance L1 is generally the longest distance between the front upper edge 60 to front bottom edge 62. Thus, in certain embodiments, while the side panels 34 and 134 extend from the waist opening 50 to one of the leg openings 52, the back side panels 134 may have a continually decreasing length dimension moving from the attachment line 66 to the distal edge 68, as is shown in FIG. 2.

Each of the side panels 34 and 134 may include one or more individual, distinct pieces of material. In particular embodiments, for example, each side panel 34 and 134 may include first and second side panel portions that are joined at a seam, with at least one of the portions comprising an elastomeric material. Still alternatively, each individual side panel 34 and 134 may include a single piece of material which is folded over upon itself along an intermediate fold line (not shown).

The side panels 34 and 134 desirably comprise an elastic material capable of stretching in a direction generally parallel to the transverse axis 49 of the diaper pant 20. In particular embodiments, the front and back side panels 34 and 134 may each comprise an interior portion 78 disposed between the distal edge 68 and the respective front or back center panel 35 or 135. In the illustrated embodiment, the interior portions 78 are disposed between the distal edges 68 and the side edges 47 of the rectangular composite structure 33. See FIG. 2. The elastic material of the side panels 34 may be disposed in the interior portions 78 to render the side panels elastomeric in a direction generally parallel to the transverse axis 49. Most desirably, each side panel 34 is elastomeric from the back bottom edge 72 to the leg end edge 70. More specifically, individual samples of side panel material, taken between the back bottom edge 72 and the leg end edge 70 parallel to the transverse axis 49 and having a length from the attachment line 66 to the distal edge 68 are all elastomeric.

Suitable elastic materials, as well as one described process of incorporating elastic side panels into a training pant, are described in the following U.S. Patents: U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, the elastic material comprises a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Mormon; and European Patent Application No. EP 0 217 032 published on Apr. 8, 1987 in the names of Taylor et al.; all of which are incorporated herein by reference. Alternatively, the side panel material may comprise other woven or nonwoven materials, such as those described above as being suitable for the outer cover 40 or bodyside liner 42, or stretchable but inelastic materials.

In a particularly preferred embodiment the side panels comprise of a material that is essentially two facing materials bonded to an elastic film material disposed between them. The elastic material is stretched to a percent elongation prior to the bonding with the facing materials, and then allowed to relax. The following patents describe how the strands are orientated in these elastic materials: U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference.

The diaper pant 20 according to the present invention may also include hook type fasteners which typically comprise a fabric or material having a base or backing structure and a plurality of hook members extending upwardly from at least one surface of the backing structure. In contrast to the loop type fasteners which desirably comprise a flexible fabric, the hook material advantageously comprises a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene or another suitable material. Suitable single-sided hook materials for the fastening components 82 and 83 or the mating fastening components 84 and 85 (FIG. 2) are available from Velcro Industries B.V., Amsterdam, Netherlands or affiliates thereof, and are identified as Velcro HTH-829 with a uni-directional hook pattern and having a thickness of about 0.089 millimeters (3.5 mils) and HTH-851 with a unidirectional hook pattern and having a thickness of about 0.051 millimeters (2 mils).

With continued reference to FIGS. 1 and 2, the side panels are generally divided into three portions, an upper portion 90, a middle portion 110 and a lower portion 100. The side panel portions generally do not comprise different materials and are not separately constructed, but rather define different regions of the side panel that may receive different treatment during manufacture. The three portions, upper 90, middle 110 and lower 100, are generally defined by their relative position within the side panel 34, 134 and in relation to the apertures 120 disposed thereon. Generally, the middle portion 110 may be defined as the portion lying between the upper and lower portions 90, 100, respectively, and bounded by the upper and lower extents of the apertures. For example, as shown in FIG. 1, the middle portion 110 is bound by box (abcd), the upper boundary (b) of which is defined by a line drawn tangent to the upper most portion of the aperture 120 and parallel to the linear end edges 45. The lower boundary (d) is defined by a line drawn tangent to the lower most portion of the aperture 120 and parallel to the linear end edges 45. The distance between upper and lower boundary (b,d) in the longitudinal direction is generally referred to the middle portion length (L4).

The middle portion 110, is generally bounded by upper and lower portions 90, 100. The upper portion 90 is generally disposed along the upper most portion of the panel 34, 134 with its upper most edge forming the waist end edge 72. The upper portion 90 is generally substantially free from apertures and has an upper portion length (L2), which is the longitudinal distance between the back bottom edge 72 and upper boundary (b) of the middle portion. The lower portion 100, lies beneath the middle portion 110 and generally extends from the lower boundary 100 and the back bottom edge 72. The lower portion 100 has a lower portion length (L3), which is the longitudinal distance between the back bottom edge 72 and the lower boundary (d) of the middle portion.

In those instances where the side panels, such as the back side panels 134 illustrated in FIG. 2 have a continually decreasing length dimension moving from the attachment line 66 to the distal edge 68, the length of the upper of lower portion is generally the shortest longitudinal distance between the upper or lower middle portion boundary and the leg or back bottom edge. For example, as illustrated in FIG. 2, the low portion longitudinal length (L2) is the shortest longitudinal distance between the middle portion boundary (b) and the front upper edge 60.

The relative lengths of L2, L3 and L4 may vary, however, in certain embodiments L4 is greater than L3, which is greater than L2. In particularly preferred embodiments L2 may range from about 25 mm to about 50 mm, such as from about 35 mm to about 45 mm and L3 may range from about 10 mm to about 25 mm, such as from about 13 mm to about 17 mm. In other embodiments, L4 may range from about from about 69 mm to about 29 mm, such as from about 47 mm to about 51 mm. Additionally, L4 is from about 28% to about 66% of L1.

Regardless of the relative lengths of L2, L3 and L4, it is generally preferred that the side panels comprise a plurality of apertures and that the apertures selectively disposed on the middle portion. The number, size and spacing of the apertures may vary depending on the desired properties of the side panel, such as optimal retention (or sustained) force at the side area of the wearer or air permeability, however, it is generally preferred that the apertures by spaced away from the waist or leg end edges. In certain embodiments the number of apertures disposed on the middle portion may range from about 69 mm to about 28 mm, such as from about 47 mm to about 51 mm such as form about 48 mm to about 50 mm. In certain embodiments, the area of the middle portion may range from about 49 mm to about 50 mm and the number of apertures per middle portion unit area may range from about 4 to about 15 slits/cm², such as from about 4 to about 10 slits/cm², such as from about 4 to about 8 slits/cm².

Just as the number of apertures disposed on the middle portion of the side panel may vary, the size and shape of the apertures may also vary. For example, as illustrated in FIG. 1, the middle portion 110 of the side panels 34, 134 may include a plurality of longitudinally oriented linear slits 120. The slits 120 may have an aspect ratio greater than about 5:1, such as greater than about 2:1, such as greater than about 1:1, when the side panel is an untensioned state.

The slits may be made by any number of well-known methods for forming an aperture in a web such as, for example, ultrasonic or thermal/pressure techniques. Such slits and forming techniques are described in U.S. Pat. No. 2,544,069 issued to Cutler on Mar. 6, 1951; and U.S. Pat. No. 4,834,738 issued to Kielpikowski et al. on May 30, 1989; each of which is incorporated herein by reference. Preferably, the slits extend through all of the layers of the stretch laminates in the side panels, such that the slits extend from the inside to the outside of the product.

Generally the slits 120 (FIG. 1) are transformed into holes 120a (FIG. 3) when the article is donned by a user and the side panels 34,134 are tensioned. Accordingly, in a preferred embodiment, the side panels 34,134 may comprise a plurality of holes selectively disposed on the middle portion thereof, where the holes have an aspect ratio greater than about 1:1. More preferably, the aspect ratio is greater than about 2:1, and can be about 3:1, 4:1, 5:1 or any other ratio greater than 1:1.

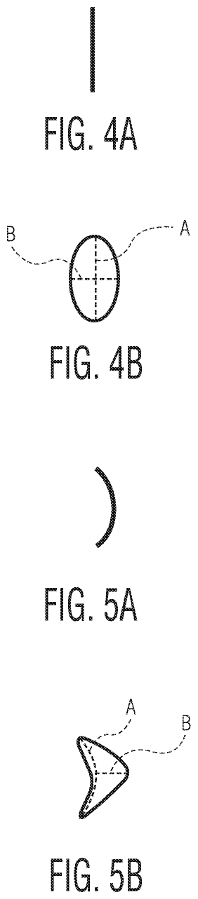

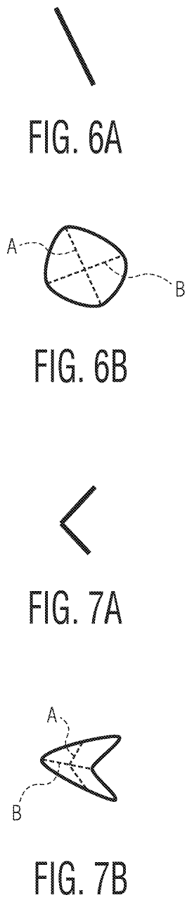

Several slit and hole shapes useful in the present invention are illustrated in FIGS. 4A-B, 5A-B, 6A-6B, and 7A-7B. As illustrated in FIGS. 4A, 5A, 6A, and 7A as the side panel containing the slits is tensioned the slit may be transformed into a hole having a shape dictated by the shape of the slit. For example, FIGS. 4B, 5B, 6B, and 7B depict several differently shaped holes having a major axis A and a minor axis B. When the slits 120 in the side panels are tensioned, the minor axis B widens, resulting in a hole that is from about 2 mm to about 5 mm and from about 3 mm to about 5 mm. Optionally, the length of each slit may also vary in size from slit to slit.

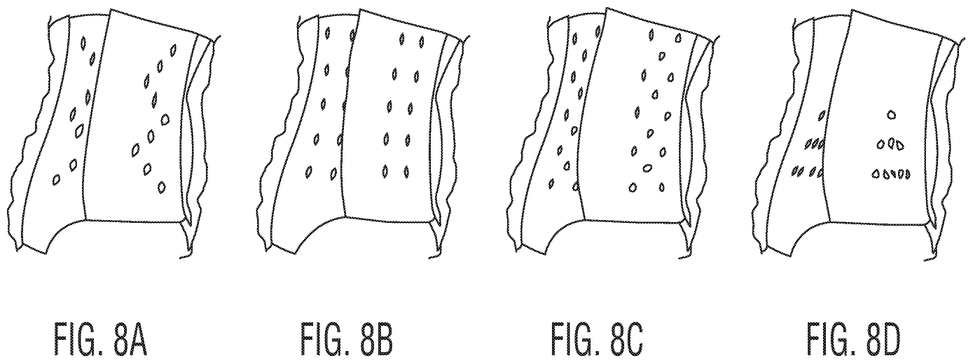

In the embodiment illustrated in FIGS. 8A-8D, examples of different aperture alignments and the patterns on a side panel of an absorbent article are shown. FIG. 8A depicts a zig-zag aperture alignment wherein the apertures do not extend to the top and bottom portions of the side panel. FIG. 8B shows diamond-like patterned apertures that are aligned parallel to each other where the apertures do not extend to the top and bottom portions of the side panel. FIG. 8C shows randomized apertures that do not extend to the top and bottom of the side panel. FIG. 8D depicts pyramid type form apertures wherein the apertures appear toward the bottom of the middle portion of the side panel but not in the top or bottom of the side panel.

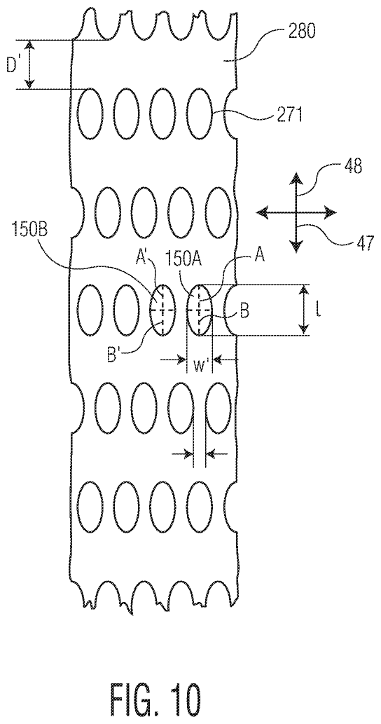

In the embodiment illustrated in FIG. 10, the holes 120a have an elliptical shape, for example, provide the benefit of retaining more open area than apertures having an aspect ratio of about 1:1 or less when the side panel is extended in a direction generally orthogonal to the major axis A of the holes 120a. Generally, when the front and back side panels 34, 134 are strained in the transverse direction, such as when the diaper pant is donned by a wearer, the major axis A shortens and the minor axis B widens and an aperture having a hole 120a is created. In this manner the holes 120a on the side panels 34, 134 may be altered between an unstrained state and strained, donned state.

When in the strained donned state the plurality of holes 120a in the front and back side panels may be parallel, perpendicular, or randomized with each individually. The holes 120a may comprise of an ordered or randomed pattern. In those instances where the holes are arranged in a non-random pattern, the pattern may include simple geometric patterns such as circles or diamonds, but also including complex patterns such as logos and trademarks.

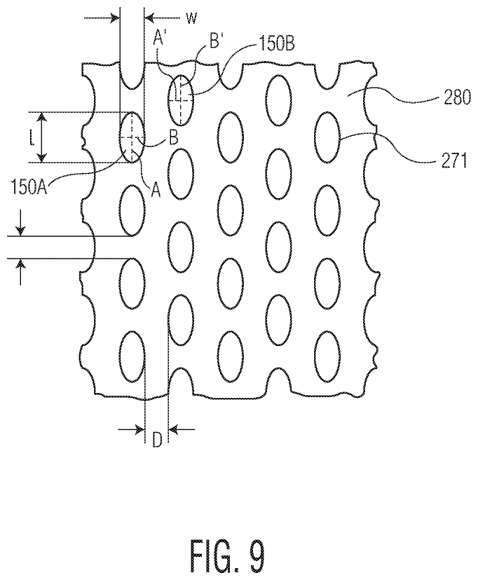

FIGS. 9 and 10 illustrate a preferred embodiment of the present invention wherein the web 280 includes a plurality of primary apertures 271 which are generally elliptical. Each primary aperture 271 has a major axis A′ and a minor axis B′ perpendicular to the major axis A′. As shown in FIG. 10, when in an untensioned state, the length (L) of the major axis A′ is longer than the length (w′) of the minor axis B′. In a preferred embodiment, the aspect ratio (length of major axis A′:length of primary axis B′) is greater than about 1.5:1. More preferably, the aspect ratio is greater than about 2:1, and can be about 3:1, 4:1, 5:1 or any other ratio greater than 1:1. The elliptical apertures 271 of the web 280 shown in FIG. 9 provide the benefit of retaining more open area than apertures having an aspect ratio of about 1:1 or less when the web 280 is extended in a direction generally orthogonal to the major axis A′ of the apertures 271. When strained, the major axis A shortens to a length of L and the minor axis lengthens to a length of w. However, due to the specific geometry of the apertures 271 and the stretch characteristics of the web 280, the reduction in percentage open area caused by the extension of the web 280 is significantly less than the reduction of open area of the web 280. Table I shows the effect geometry has on the open area measurements of strained and unstrained webs.

Another factor which may affect the percentage open area, and thus, breathability of the web 280 of the present invention is the pattern of the primary apertures 271. Specifically, the pattern can influence the resultant open area as a function of a percentage strain. Thus, a certain pattern of apertures such as the staggered pattern of apertures 271 shown in FIG. 9 or the close knit parallel patterned apertures 271 shown in FIG. 10 may provide the web 280 with a greater percentage open area when stretched than a less loosely packed or otherwise differently oriented pattern of apertures.

While the patterns illustrated in FIGS. 9 and 10 comprise apertures 271 of similar shape and size, the invention is not so limited. In certain instances the apertures may be of different shape, or of different size, or different shape and size. For example, the width may be from about 2 millimeters to about 5 millimeters. In FIG. 10 , w′ is the width of a primary hole 271, wherein the primary hole 271 width may be from about 2 to about 5 millimeters. Further, the spacing between apertures, whether within a motif or between the motifs of a pattern, need not be the same. The transverse distance between the apertures may be from the range of 1 to 5 millimeters.

Regardless of the particularly pattern or size and shape of apertures, the side panels of the present invention have improved air permeability over side panels of the prior art. Generally, the apertures on the front and back side panels permit the passage of air and water vapor to and from the interior of the diaper pant. In this configuration, exudates are prevented from leaking out of the areas adjacent the absorbent core but air and water vapor are allowed to be exchanged in the product to ventilate it so that the product does not become excessively wetted by body perspiration and uncomfortable to wear. As shown in the table below, diaper pants according to the present invention generally have improved air permeability in both a tensioned and untensioned state.

TABLE 1

| | Invention diaper | Current diaper | |
|---|---|---|---|
| | pant | pant | Pampers ® |
| Untensioned Air | 83.8 | 64.4 | 390 |
| Permeability (cfm) | | | |
| Tensioned Air | 370 | 290 | 409 |
| Permeability (cfm) | | | |

Accordingly, the present invention provides a side panel having an air permeability of at least about 67 cfm, such as at least about 83.8 cfm, such as at least about 89 cfm, such as from about 67 to about 89 cfm when in an untensioned state. Further, because the invention provides apertures that change in shape and area when tensioned, the side panels of the present invention have increased air permeability when in a tensioned state. For example, the side panels may have a tensioned air permeability of at least about 312 cfm, such as at least about 370 cfm, such as at least about 388 cfm, such as from about 312 to about 388 cfm. In other instances the panels may have a tensioned air permeability that is at least about 80% greater, such as at least about 80% greater than the untentionsed air permeability.

In other instances the aperture side panels of the present invention provide the benefit of reducing wearer leg load. The side panel preferably has peak load of at least 600 gf. A side panel having a peak load ranging from about 600 gf to about 700 gf, provides a garment that is easily donned by a wearer, yet provides a suitable level of body fitting/conformity. Further, a side panel having a peak load ranging from about 600 gf to about 700 gf tends not to cause red marking on the skin of the wearer and generally does not cause discomfort for the wearer during usage. Thus, the side panels of the present invention may be incorporated into absorbent articles, particularly diapers and the like, that provide enough elastic force to keep the article in place on the wearer and provide good body fit.

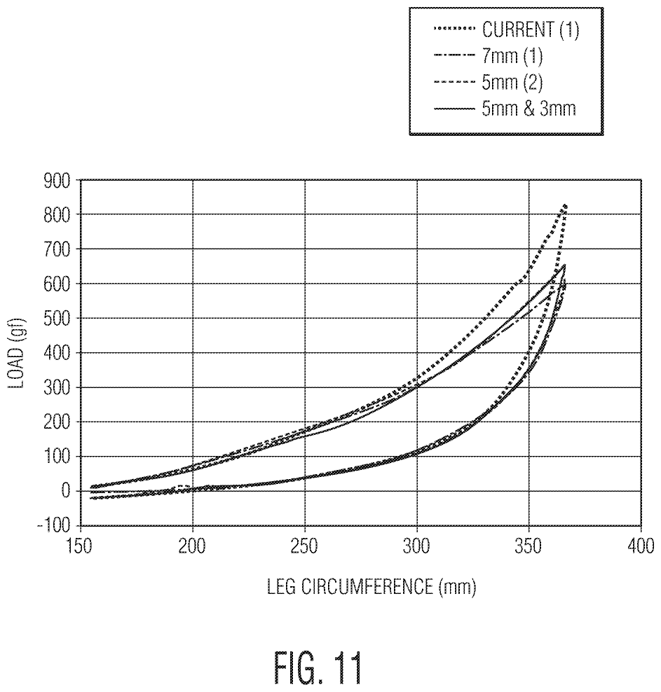

FIG. 11 shows one preferred example of the extension and recovery force curves for the two cycle hysteresis of side panels prepared according to the present invention. In preferred embodiments, the peak load is at least 700 gf. The First Cycle Extension Force is the amount of force it takes to reduce stretch loads around the waist and legs of the wearer.

### Test Methods

### Air Permeability

Air permeability was tested with a known FX3300 instrument. The Air Permeability FX3300 Tester is used for fast, simple, and accurate determination of the air permeability of non-wovens and forming fabrics. Air permeability measured in CFM or l/dm2.min, is determined by applying a very small pressure difference (of about 0.018-0.029 PSI or 1.27-2 mbar) between the two sides of a fabric and then measuring how much air flows through the fabric. For example, in the case of a very fine, dense fabric less air will pass through and thus it will have a lower CFM value.

### Embodiments:

1. An absorbent article (20), comprising:
a. an absorbent chassis (32)defining:
   i. a longitudinal axis (48), a transverse axis(49), front and back waist edges parallel to the transverse axis, opposite side edges extending between the front and back waist edges,
   ii. a front waist region (22) contiguous with the front waist edge,
   iii. a back waist region (24) contiguous with the back waist edge, and
   iv. a crotch region (26) which extends between and interconnects the front and back waist regions,
      1. the front waist region (22) defining a pair of transversely opposed front side panels (34) having an upper edge (60) contiguous with the front waist edge and a bottom edge (62) and a front center panel positioned between and interconnecting the front side panels,
      2. the back waist region defining a pair of transversely opposed back side panels (134) having an upper edge (70) contiguous with the back waist edge and a bottom edge (72) and a back center panel positioned between and interconnecting the back side panels,
   wherein the front and back side (34, 134) panels have a length (L1) extending from the upper edge to the lower edge, an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4)
      3. a plurality of slits (120) disposed within the middle portion and wherein the upper and lower portions are substantially free from slits.
2. The absorbent article (20) according to claim 1, wherein the front side panels and the back side panels have a first outer surface and a opposite second outer surface and wherein the plurality of slits extend from the first outer surface to the second outer surface.
3. The absorbent article (20) according to claim 1, wherein the slits have an aspect ratio, and the aspect ratio decreases when the article is donned.
4. The absorbent article (20) according to claim 1, wherein the slits are arranged in longitudinally extending rows, wherein the rows of slits are spaced apart and parallel to one another.
5. The absorbent article (20) according to claim 1, wherein the slits are arranged in transversely extending rows, wherein the rows of slits are spaced apart and parallel to one another.
6. The absorbent article (20) according to claim 1, wherein the slits are arranged in transversely extending rows, wherein the rows of slits are spaced apart and perpendicular to one another,
7. The absorbent article (20) according to claim 1, wherein the transverse spacing between slits is substantially equal.
8. The absorbent article (20) according to claim 1, wherein the longitudinal spacing between slits is substantially equal.
9. The absorbent article (20) according to claim 1, wherein all of the slits are similarly shaped and have substantially similar aspect ratios.
10. The absorbent article (20) according to claim 1, wherein each of the plurality of slits are spaced apart from one another in the longitudinal direction at least about 5.0 mm.
11. The absorbent article (20) according to claim 1, wherein each of the plurality of slits are spaced apart from one another in the transverse direction at least about 5.0 mm.
12. The absorbent article (20) according to claim 1, wherein all of the slits are similarly shaped and have substantially similar aspect ratios.
13. The absorbent article (20) according to claim 1, wherein L2 is from about 24% to about 48% of the front and back side panel length, L3 is about 10% to about 24% of the front and back side panel length and L4 is about 28% to about 66% of the front and back side panel length.
14. The absorbent article (20) according to claim 1, wherein L2 has a length of about 40 mm and L3 has a length of about 15 mm.
15. The absorbent article (20) according to claim 1 wherein L4 is greater than L2 and L3.
16. The absorbent article (20) according to claim 1, wherein L2 is at least about 36% greater than L3.
17. The absorbent article (20) according to claim 1, wherein the slits (120) are disposed in a pattern.
18. The absorbent article (20) according to claim 17, wherein the pattern is curvilinear, linear, cross, or convergent.
19. The absorbent article (20) according to claim 1, wherein the front and back side panels are moved in a MD direction.
20. The absorbent article (20) according to claim 1, wherein the front and back side panels comprises a single layer of elastomeric material.
21. The absorbent article (20) according to claim 1, wherein the front side panels and the back side panels have an air permeability greater than about 67 cfm.
22. The absorbent article (20) according to claim 1, wherein the front side panels and the back side panels have a First Cycle Extension Force less than about 700 gf.
23. An absorbent article (20), comprising:
   a. an absorbent chassis (32) defining:
      i. a longitudinal axis (48), a transverse axis(49), front and back waist edges parallel to the transverse axis, opposite side edges extending between the front and back waist edges,
      ii. a front waist region (22) contiguous with the front waist edge,
      iii. a back waist region (24) contiguous with the back waist edge, and
      iv. a crotch region (26) which extends between and interconnects the front and back waist regions,
         1. the front waist region (22) defining a pair of transversely opposed front side panels (34) having an upper edge (60) contiguous with the front waist edge and a bottom edge (62) and a front center panel positioned between and interconnecting the front side panels,
         2. the back waist region defining a pair of transversely opposed back side panels (134) having an upper edge (70) contiguous with the back waist edge and a bottom edge (72) and a back center panel positioned between and interconnecting the back side panels,
         wherein the front and back side (34, 134) panels have a length (L1) extending from the upper edge to the bottom edge, an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4)
         3. a plurality of slits (120) disposed within the middle portion wherein the upper and lower portions are substantially free from slits and wherein the slits (120) become holes (120a) when the absorbent article is donned.
24. The absorbent article (20) according to claim 23, wherein the holes (120a) are disposed in a pattern.
25. The absorbent article (20) according to claim 24, wherein the pattern may be straight lines, curvy lines, angles, circles, squares, stars, intersecting lines, rectangles, triangles, octagons, animals, teddy bears, clouds, signs, logos, human, animal or animated characters or any other similar design pattern thereof.
26. The absorbent article (20) according to claim 23, wherein the slits have an aspect ratio, and the aspect ratio decreases when the article is donned.
27. The absorbent article (20) according to claim 23, wherein the slits are arranged in longitudinally extending rows, wherein the rows of slits are spaced apart and parallel to one another.
28. The absorbent article (20) according to claim 23, wherein the slits are arranged in transversely extending rows, wherein the rows of slits are spaced apart and parallel to one another.
29. The absorbent article (20) according to claim 23, wherein the slits are arranged in transversely extending rows, wherein the rows of slits are spaced apart and perpendicular to one another
30. The absorbent article (20) according to claim 23, wherein the transverse spacing between slits is substantially equal.
31. The absorbent article (20) according to claim 23, wherein the longitudinal spacing between slits is substantially equal.
32. The absorbent article (20) according to claim 23, wherein all of the slits are similarly shaped and have substantially similar aspect ratios.
33. The absorbent article (20) according to claim 23, wherein each of the plurality of slits are spaced apart from one another in the longitudinal direction at least about 5.0 mm.
34. The absorbent article (20) according to claim 23, wherein each of the plurality of slits are spaced apart from one another in the transverse direction at least about 5.0 mm.
35. The absorbent article (20) according to claim 23, wherein all of the slits are similarly shaped and have substantially similar aspect ratios.
36. The absorbent article (20) according to claim 23, wherein L2 is from about 24% to about 48% of the front and back side panel length, L3 is about 10% to about 24% of the front and back side panel length and L4 is about 28% to about 66% of the front and back side panel length.
37. The absorbent article (20) according to claim 23, wherein L2 has a length of about 40 mm and L3 has a length of about 15 mm.
38. The absorbent article (20) according to claim 23, wherein L4 is greater than L2 and L3.
39. The absorbent article (20) according to claim 23, wherein L2 is at least about 36% greater than L3.
40. The absorbent article (20) according to claim 23, wherein the slits (120) are disposed in a pattern.
41. The absorbent article (20) according to claim 40, wherein the pattern is curvilinear, linear, cross, or convergent.
42. The absorbent article (20) according to claim 23, wherein the front and back side panels are moved in a MD direction.
43. The absorbent article (20) according to claim 23, wherein the front and back side panels comprises a single layer of elastomeric material.
44. The absorbent article (20) according to claim 23, wherein the front side panels and the back side panels have an air permeability greater than about 312 cfm when the absorbent article is donned.
45. The absorbent article (20) according to claim 23, wherein the front side panels and the back side panels have a First Cycle Extension Force less than about 700 gf.
46. An absorbent article (20) comprising:
   a chassis (32) having a longitudinal axis(48), a transverse axis(49), a front waist region (22) having a front upper edge (60) and a front bottom edge (62) and a back waist region (24) having a back upper edge (70) and a back bottom edge (72);
   a pair of elastomeric front side panels that are extensible in the transverse direction, the front side panels extending from the front waist portion and having an upper edge portion that is contiguous with the front waist edge, and a lower edge portion, an outer most surface and opposed inner most surface, each front side panel having a length (L1) extending from the upper edge to the lower edge, an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4), wherein the middle portion comprises a plurality of longitudinally orientated slits extending from the outer most surface to the inner most surface and having a first aspect ratio and the upper and lower portions are substantially free from slits; and
   a pair of elastomeric back side panels that are extensible in the transverse direction, the front side panels extending from the back waist region (24) and having a front upper edge (60) that is contiguous with the front bottom edge (62), and a back bottom edge (72), an outer most surface and opposed inner most surface, each back side panel having a length (L1) extending from the upper edge (60) to the bottom edge (62), an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4), wherein the middle portion comprises a plurality of longitudinally orientated slits extending from the outer most surface to the inner most surface and having a first aspect ratio and the upper and lower portions are substantially free from slits (120), wherein upon donning of the article by a wearer the first aspect ratio is decreased.
47. The absorbent article (20) according to claim 46, wherein the slits are arranged in longitudinally extending rows, wherein the rows of slits are spaced apart and parallel to one another.
48. The absorbent article (20) according to claim 46, wherein the slits are arranged in transversely extending rows, wherein the rows of slits are spaced apart and parallel to one another.
49. The absorbent article (20) according to claim 46, wherein the slits are arranged in transversely extending rows, wherein the rows of slits are spaced apart and perpendicular to one another.
50. The absorbent article (20) according to claim 46, wherein the transverse spacing between slits is substantially equal.
51. The absorbent article (20) according to claim 46, wherein the longitudinal spacing between slits is substantially equal.
52. The absorbent article (20) according to claim 46, wherein all of the slits are similarly shaped and have substantially similar aspect ratios.
53. The absorbent article (20) according to claim 46, wherein each of the plurality of slits are spaced apart from one another in the longitudinal direction at least about 5.0 mm.
54. The absorbent article (20) according to claim 46, wherein each of the plurality of slits are spaced apart from one another in the transverse direction at least about 5.0 mm.
55. The absorbent article (20) according to claim 46, wherein all of the slits are similarly shaped and have substantially similar aspect ratios.
56. The absorbent article (20) according to claim 46, wherein L2 is from about 24% to about 48% of the front and back side panel length, L3 is about 10% to about 24% of the front and back side panel length and L4 is about 28% to about 66% of the front and back side panel length.
57. The absorbent article (20) according to claim 46, wherein L2 has a length of about 40 mm and L3 has a length of about 15 mm.
58. The absorbent article (20) according to claim 46, wherein L4 is greater than L2 and L3.
59. The absorbent article (20) according to claim 46, wherein L2 is at least about 36% greater than L3.
60. The absorbent article (20) according to claim 46, wherein the slits (120) are disposed in a pattern.
61. The absorbent article (20) according to claim 46, wherein the pattern is curvilinear, linear, cross, or convergent.
62. The absorbent article (20) according to claim 46, wherein the front and back side panels are moved in a MD direction.
63. The absorbent article (20) according to claim 46, wherein the front and back side panels comprises a single layer of elastomeric material.
64. The absorbent article (20) according to claim 46, wherein the front side panels and the back side panels have an air permeability greater than about 312 cfm when the absorbent article is donned.
65. The absorbent article (20) according to claim 46, wherein the front side panels and the back side panels have a First Cycle Extension Force less than about 700 gf.
66. A side panel for joining the front waist region (22) and rear waist region (24) of an absorbent article chassis(32) having a longitudinal axis (48) and a transverse axis (49), the side panel comprising:
   an elastic laminate extensible in the transverse direction and comprising two layers of nonwoven on either side of an elastic film, the laminate having first and second opposed outer surfaces;
   an upper edge;
   a lower edge;
   a length (L1) extending from the upper edge to the lower edge;
   an upper portion (90) adjacent to the upper edge (60), (70) and having an upper portion length (L2) and being substantially free from apertures,
   a lower portion (100) adjacent to the bottom edge (62), (72) and having a lower portion length (L3) and being substantially free from apertures, and a
   middle portion (110) disposed between the upper and lower portions and having a middle portion length (L4) and having a plurality of longitudinally orientated slits (120) extending from the first to the second outer surfaces and having a first aspect ratio.
67. The absorbent article (20) according to claim 66, wherein the slits have an aspect ratio, and the aspect ratio decreases when the article is donned.
68. The absorbent article (20) according to claim 66, wherein the slits are arranged in longitudinally extending rows, wherein the rows of slits are spaced apart and parallel to one another.
69. The absorbent article (20) according to claim 66, wherein the slits are arranged in transversely extending rows, wherein the rows of slits are spaced apart and parallel to one another.
70. The absorbent article (20) according to claim 66, wherein the slits are arranged in transversely extending rows, wherein the rows of slits are spaced apart and perpendicular to one another.
71. The absorbent article (20) according to claim 66, wherein the transverse spacing between slits is substantially equal.
72. The absorbent article (20) according to claim 66, wherein the longitudinal spacing between slits is substantially equal.
73. The absorbent article (20) according to claim 66, wherein all of the slits are similarly shaped and have substantially similar aspect ratios.
74. The absorbent article (20) according to claim 66, wherein each of the plurality of slits are spaced apart from one another in the longitudinal direction at least about 5.0 mm.
75. The absorbent article (20) according to claim 66, wherein each of the plurality of slits are spaced apart from one another in the transverse direction at least about 5.0 mm.
76. The absorbent article (20) according to claim 66, wherein all of the slits are similarly shaped and have substantially similar aspect ratios.
77. The absorbent article (20) according to claim 66, wherein L2 is from about 24% to about 48% of the front and back side panel length, L3 is about 10% to about 24% of the front and back side panel length and L4 is about 28% to about 66% of the front and back side panel length.
78. The absorbent article (20) according to claim 66, wherein L2 has a length of about 40 mm and L3 has a length of about 15 mm.
79. The absorbent article (20) according to claim 66, wherein L4 is greater than L2 and L3.
80. The absorbent article (20) according to claim 66, wherein L2 is at least about 36% greater than L3.
81. The absorbent article (20) according to claim 66, wherein the slits (120) are disposed in a pattern.
82. The absorbent article (20) according to claim 66, wherein the pattern is curvilinear, linear, cross, or convergent.
83. The absorbent article (20) according to claim 66, wherein the front and back side panels are moved in a MD direction.
84. The absorbent article (20) according to claim 66, wherein the front and back side panels comprises a single layer of elastomeric material.
85. The absorbent article (20) according to claim 66, wherein the front side panels and the back side panels have an air permeability greater than about 67 cfm.
86. The absorbent article (20) according to claim 66, wherein the front side panels and the back side panels have a First Cycle Extension Force less than about 700 gf.

1. An absorbent article (20), comprising:
a. an absorbent chassis (32)defining:
i. a longitudinal axis (48), a transverse axis(49), front and back waist edges parallel to the transverse axis, opposite side edges extending between the front and back waist edges,
ii. a front waist region (22) contiguous with the front waist edge,
iii. a back waist region (24) contiguous with the back waist edge, and
iv. a crotch region (26) which extends between and interconnects the front and back waist regions,
1. the front waist region (22) defining a pair of transversely opposed front side panels (34) having an upper edge (60) contiguous with the front waist edge and a bottom edge (62) and a front center panel positioned between and interconnecting the front side panels,
2. the back waist region defining a pair of transversely opposed back side panels (134) having an upper edge (70) contiguous with the back waist edge and a bottom edge (72) and a back center panel positioned between and interconnecting the back side panels,
wherein the front and back side (34, 134) panels have a length (L1) extending from the upper edge to the lower edge, an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4)
3. a plurality of slits (120) disposed within the middle portion and wherein the upper and lower portions are substantially free from slits.

2. The absorbent article (20) according to claim 1, wherein the front side panels and the back side panels have a first outer surface and a opposite second outer surface and wherein the plurality of slits extend from the first outer surface to the second outer surface.

3. The absorbent article (20) according to claim 1, wherein the slits have an aspect ratio, and the aspect ratio decreases when the article is donned.

4-9. (canceled)

10. The absorbent article (20) according to claim 1, wherein each of the plurality of slits are spaced apart from one another in the longitudinal direction at least about 5.0 mm.

11. The absorbent article (20) according to claim 1, wherein each of the plurality of slits are spaced apart from one another in the transverse direction at least about 5.0 mm.

12. (canceled)

13. The absorbent article (20) according to claim 1, wherein L2 is from about 24% to about 48% of the front and back side panel length, L3 is about 10% to about 24% of the front and back side panel length and L4 is about 28% to about 66% of the front and back side panel length.

14. (canceled)

15. The absorbent article (20) according to claim 1 wherein L4 is greater than L2 and L3.

16. The absorbent article (20) according to claim 1, wherein L2 is at least about 36% greater than L3.

17-22. (canceled)

23. An absorbent article (20), comprising:
a. an absorbent chassis (32) defining:
i. a longitudinal axis (48), a transverse axis(49), front and back waist edges parallel to the transverse axis, opposite side edges extending between the front and back waist edges,
ii. a front waist region (22) contiguous with the front waist edge, viii. a back waist region (24) contiguous with the back waist edge, and
iv. a crotch region (26) which extends between and interconnects the front and back waist regions,
1. the front waist region (22) defining a pair of transversely opposed front side panels (34) having an upper edge (60) contiguous with the front waist edge and a bottom edge (62) and a front center panel positioned between and interconnecting the front side panels,
2. the back waist region defining a pair of transversely opposed back side panels (134) having an upper edge (70) contiguous with the back waist edge and a bottom edge (72) and a back center panel positioned between and interconnecting the back side panels,
wherein the front and back side (34, 134) panels have a length (L1) extending from the upper edge to the bottom edge, an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4)
3. a plurality of slits (120) disposed within the middle portion wherein the upper and lower portions are substantially free from slits and wherein the slits (120) become holes (120a) when the absorbent article is donned.

24-25. (canceled)

26. The absorbent article (20) according to claim 23, wherein the slits have an aspect ratio, and the aspect ratio decreases when the article is donned.

27-35. (canceled)

36. The absorbent article (20) according to claim 23, wherein L2 is from about 24% to about 48% of the front and back side panel length, L3 is about 10% to about 24% of the front and back side panel length and L4 is about 28% to about 66% of the fro nt and back side panel length.

37. The absorbent article (20) according to claim 23, wherein L2 has a length of about 40 mm and L3 has a length of about 15 mm.

38-43. (canceled)

44. The absorbent article (20) according to claim 23, wherein the front side panels and the back side panels have an air permeability greater than about 312 cfm when the absorbent article is donned.

45. The absorbent article (20) according to claim 23, wherein the front side panels and the back side panels have a First Cycle Extension Force less than about 700 gf.

46. An absorbent article (20) comprising:
a chassis (32) having a longitudinal axis(48), a transverse axis(49), a front waist region (22) having a front upper edge (60) and a front bottom edge (62) and a back waist region (24) having a back upper edge (70) and a back bottom edge (72);
a pair of elastomeric front side panels that are extensible in the transverse direction, the front side panels extending from the front waist portion and having an upper edge portion that is contiguous with the front waist edge, and a lower edge portion, an outer most surface and opposed inner most surface, each front side panel having a length (L1) extending from the upper edge to the lower edge, an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4), wherein the middle portion comprises a plurality of longitudinally orientated slits extending from the outer most surface to the inner most surface and having a first aspect ratio and the upper and lower portions are substantially free from slits; and
a pair of elastomeric back side panels that are extensible in the transverse direction, the front side panels extending from the back waist region (24) and having a front upper edge (60) that is contiguous with the front bottom edge (62), and a back bottom edge (72), an outer most surface and opposed inner most surface, each back side panel having a length (L1) extending from the upper edge (60) to the bottom edge (62), an upper portion (90) adjacent to the upper edge and having an upper portion length (L2), a lower portion (100) adjacent to the bottom edge and having a lower portion length (L3), and a middle portion (110) disposed between the upper and lower portions and having a middle portion length (L4), wherein the middle portion comprises a plurality of longitudinally orientated slits extending from the outer most surface to the inner most surface and having a first aspect ratio and the upper and lower portions are substantially free from slits (120), wherein upon donning of the article by a wearer the first aspect ratio is decreased.

47-55. (canceled)

56. The absorbent article (20) according to claim 46, wherein L2 is from about 24% to about 48% of the front and back side panel length, L3 is about 10% to about 24% of the front and back side panel length and L4 is about 28% to about 66% of the front and back side panel length.

57. The absorbent article (20) according to claim 46, wherein L2 has a length of about 40 mm and L3 has a length of about 15 mm.

58. The absorbent article (20) according to claim 46, wherein L4 is greater than L2 and L3.

59. The absorbent article (20) according to claim 46, wherein L2 is at least about 36% greater than L3.

60-62. (canceled)

63. The absorbent article (20) according to claim 46, wherein the front and back side panels comprises a single layer of elastomeric material.

64-86. (canceled)
